(19) United States
(12) Patent Application Publication
(10) Pub. No.: US 2025/0127660 A1
(43) Pub. Date: Apr. 24, 2025

(54) Knife for goniotomy

(71) Applicant: Zhongshan Ophthalmic Center of Sun Yat-sen University, Guangzhou (CN)

(72) Inventors: Xiulan ZHANG, Guangzhou (CN); Fengbin LIN, Guangzhou (CN); Yunhe SONG, Guangzhou (CN)

(21) Appl. No.: 18/492,949

(22) Filed: Oct. 24, 2023

(51) Int. Cl. A61F 9/007; A61B 17/00; A61B 17/32; A61B 17/3211

(52) U.S. Cl. CPC A61F 9/00772; A61B 2017/00424; A61B 17/320016; A61B 17/3211

(57) ABSTRACT

The present invention discloses a knife for GT, including a handle portion, at least one end of the handle portion being sequentially connected to a neck portion and a head portion, wherein the neck portion is smoothly transited to the head portion and formed an angle of 85-95 degrees therebetween, the head portion has a blunt tip end, and a diameter of the head portion is decreased towards the blunt tip end to form a knife face. The knife of the present invention is convenient to use and easy to operate, more conforming to the requirements of the ergonomics, and thus achieves improved operation efficiency.

### TECHNICAL FIELD

The present invention relates to the technical field of medical instruments, and particularly relates to a knife for goniotomy (GT).

### BACKGROUND

Glaucoma is the leading irreversible cause of blindness all over the world. Currently, with the increasing development and innovation of the glaucoma diagnosis and treatment technology, glaucoma surgery has gradually changed from traditional filtering surgery, which is complicated to operate and causes more complications, to minimally invasive glaucoma surgery (MIGS), which is more in line with the physiological structure of eyes, easy to operate and may cause fewer complications.

Among them, GT, also known as ab interno trabeculotomy, a type of MIGS, has been proved to be an effective method for treating glaucoma. It can effectively reduce intraocular pressure by incising the diseased trabecular meshwork to increase outflow of the aqueous humor.

Clinically, there are various surgical instruments for GT, including Kahook Dual Blade (KDB), Tanito Microhook (TMH), retinal curettage hook, and a knife made by bending 25G 1 ml syringe needle or 26G vesicle breaking needle. However, no matter which kind of instruments are available, it is unable to effectively and conveniently achieve incising the trabecular meshwork in minimally invasive method, which brings inconvenience for clinical surgery.

For example, the disadvantages of KDB or retinal curettage hook for GT are that wide tip of the head thereof may damage to the tissue around the anterior chamber angle, as the anterior chamber angle of humans, especially for Asian, is always narrow; and the possible incising range is limited, mostly only 120 degrees can be incised.

The disadvantages of TMH for GT are that the relatively short knife shaft may makes the knife head cannot be enough to attach the trabecular meshwork for trabeculotomy, in a case that eyeballs of the patient or hands of the operator are relatively big, especially for one wearing gloves more than No. 7.5.

Regarding the knife made by bending 25G 1 ml syringe needle or 26G vesicle breaking needle for GT, sharp knife head may damage to the rear wall of the Schlemm canal and the tissue around the anterior chamber angle, as a result of bleeding.

It is therefore desirable to provide a specialized knife for GT, which can be used for GT in MIGS.

### SUMMARY

The present invention therefore seeks to provide a knife for GT for incising the trabecular meshwork of an eye, which is more convenient and standardized and is capable of improving surgical efficiency and thus achieving high operation safety.

According to the present invention, a knife for GT includes a handle portion, at least one end of the handle portion being sequentially connected to a neck portion and a head portion. The neck portion is smoothly transited to the head portion and formed an angle of 85-95 degrees. The head portion of the present invention is ended with a blunt tip, and the diameter of the head portion is decreased towards the tip end to form the knife face.

The head portion, neck portion and the handle portion may be integrally formed and made of stainless-steel material. The neck portion and the handle portion may be in substantially cylindrical shape.

As the anterior chamber angle of the eye is very small, in order to avoid accident injury to the tissue around the anterior chamber angle, the head portion may be designed into a cylindrical shape with the blunt tip, including a cylindrical segment smoothly transited to the neck portion, thereby achieving incising the trabecular meshwork. The diameter of the tip is designed so that the anterior chamber angle can be incised, and especially designed to be blunt as possible to avoid accident injury to other portions.

As such, according to the present invention, the total length of the head portion may range from 0.6 mm to 1.0 mm, and/or the diameter of the cylindrical segment of the head portion may range from 0.4 mm to 0.6 mm. Such design of the head portion can be applicable to incise the trabecular meshwork.

According to the present invention, the length of the head portion is preferably 0.7 mm, 0.8 mm or 0.9 mm; the diameter of the cylindrical segment of the head portion is preferably 0.4 mm, 0.5 mm or 0.6 mm; and/or the angle of between the head portion and the neck portion is preferably 89 degrees, 90 degrees or 91 degrees.

According to an embodiment of the present invention, the neck portion may be in form of an elongated shape with a length of 22-30 mm. The front end of the neck portion is connected to the head portion, the rear end of the neck portion is connected to the handle portion, and the diameter of the neck portion is gradually increased from the front end to the rear end thereof.

Particularly, the neck portion is formed in a columnar shape with diameter gradually increased from the front end to the rear end. Such configuration is advantageous for the operators to exert force and thus to easily incise the trabecular meshwork. The length of the neck portion is preferably 25 mm, 26 mm, or 27 mm according to the present invention.

According to another embodiment of the present invention, the neck portion of the goniotomy knife may be designed in a bending shape, including a first transition part connected to the head portion and a second transition part connected to the handle portion, with the first transition part and the second transition part forming a bending angle of 125-145 degrees.

The bending angle between the first transition part and the second transition part is preferably 130 degrees, 131 degrees, 132 degrees, 133 degrees, 134 degrees, 135 degrees, 136 degrees, 137 degrees, 138 degrees, 139 degrees, or 140 degrees.

Particularly, the second transition part is preferably in a line with the handle portion, and the first transition part may be upwardly or downwardly bended relative to the second transition part. The head portion thus is inclined upwardly or downwardly relative to the first transition part 210 at an angle.

The length of the first transition part may be 9-13 mm, the length of the second transition part may be 13-17 mm, and/or the diameter of the neck portion is gradually increased from the first transition part to the second transition part.

According to the present invention, the length of the first transition part is preferably 10 mm, 11 mm or 12 mm, and the length of the second transition part is preferably 14 mm, 15 mm, or 16 mm.

According to the present invention, there may be head portion and neck portion at both ends of the handle portion.

In this case, the neck portion at both ends of the handle portion may all be in elongated shape, or also be in bending shape, alternatively, the neck portion at one end of the handle portion is in elongated shape, while the neck portion at the other end of the handle portion is in bending shape.

In the present invention, the length of the handle portion may be 70-130 mm.

In the case that only one end of the handle portion is connected with the neck portion and the head portion, the handle portion can be designed longer for grabbing, preferably 100-115 mm. While in the case that both ends of the handle portion are connected with the neck portion and the head portion, the handle portion can be designed shorter to avoid too long overall length, and preferably 70-100 mm.

According to the present invention, the handle portion includes an operation part for grabbing and a joining part connected to the neck portion. The operation part is preferably in form of cylinder and the joining part is preferably in form of circular truncated cone. The end of the joining part connected to the neck portion is smaller than the other end thereof.

In the present invention, the operation part may be provided with a plurality of projections, and a plurality of threaded structures may be provided between the adjacent projections.

The threaded structures can increase friction of the operation part, which is better for the operators to grab the knife. According to the present invention, the projections and the threaded structures may be formed by rubber sleeves or rubber rings sleeved on the operation part, or formed by stainless-steel material integrally formed with the operation part.

Alternatively, the joining part may be detachably connected to the operation part, namely, the neck portion is detachably connected to the operation part. In such easy way, the neck portion can be displaced for cutting open the anterior chamber angle of the eye at different situation. Particularly, the joining part is connected to the operation part by screws. In this case, the joining part, the neck portion and the head portion may be integrally formed.

It should be noted that when both ends of the handle portion are connected with the neck portion, both ends of the operation part are correspondingly provided with the joining part. While in the case that one end of the handle portion is connected with the neck portion, only one end of the operation part is correspondingly provided with the joining part, or both ends of the operation part can be correspondingly provided with the joining part.

Compared to the prior art, the knife for GT includes a head portion, a neck portion and a handle portion, wherein the head portion is formed in substantially cylindrical shape with a blunt tip end, thereby prevent bleeding caused by blood vessel rupture due to maloperation during surgery and tip end making the anterior chamber angle of the eye successfully cut open. The angle between the head portion the elongated or bending neck portion can facilitate the head portion of the knife entering the surgery section and be better applicable for surgery in different portion. The handle portion in the present invention designed into elongated columnar shape is convenient for the operators to grasp and thus flexibly use the knife. In addition, in the case that both ends of the handle portion are provided with the neck portion and the head portion, operators can select suitable head portion to operate, there is no need to displace the other knife during surgery, thus improving operation efficiency and adaptability of the knife. Therefore, the knife of the present invention is convenient to use and easy to operate, more conforming to the requirements of the ergonomics, and achieves improved operation efficiency.

### DESCRIPTION OF THE DRAWINGS

FIG. 1 is a structural schematic view of a knife according to an embodiment of the present invention;

FIG. 2 is a structural schematic view of a knife according to another embodiment of the present invention; and

FIG. 3 is an enlarged view of a head portion of FIG. 1 and FIG. 2.

### DETAILED DESCRIPTION

The accompanying drawings are for exemplary illustration only, and should not be construed as limitations on the present invention. In order to better illustrate the following embodiment, some parts in the accompanying drawings may be omitted, enlarged or reduced, and they do not represent the size of the actual product. For those skilled in the art, it is understandable that certain well-known structures and descriptions thereof in the drawings may be omitted.

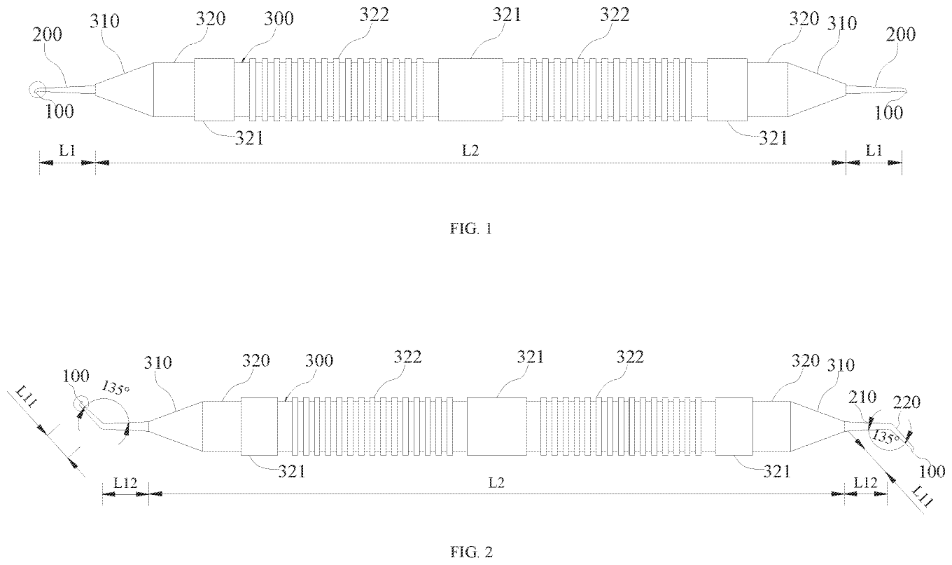

FIG. 1 depicts a knife for GT according to an embodiment of the present invention, which is used to incise the trabecular meshwork of an eye, having a handle portion 300, at least one end of the handle portion 300 being sequentially connected to a neck portion 200 and a head portion 100. The head portion 100, neck portion 200 and the handle portion 300 in this embodiment are integrally formed and made of stainless-steel material. Stainless-steel material facilitates sterilization at high temperature and reuse, thus reducing the waste of medical consumables. The neck portion 200 and the handle portion 300 may be in substantially cylindrical shape, such configuration conforms to the requirements of the ergonomics and is thus good for the wrist or fingers of the ophthalmologists to exert force conveniently.

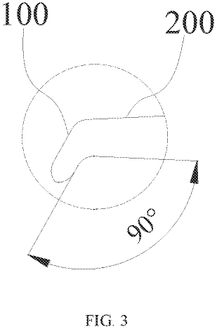

Particularly, the head portion 100 extends downwardly relative to the neck portion 200. That is, the head portion 100 and the neck portion 200 forms an angle, especially 90 degrees, as FIG. 3 shown. However, in the context of the present invention, the angle between the head portion 100 and the neck portion 200 is 85-95 degrees. In order to avoid accidental injury to the tissue around anterior chamber angle of an eye, the neck portion 200 is smoothly transited to the head portion 100. The head portion 100 is in substantially cylindrical form while with a tip, namely, the head portion 100 has a substantially cylindrical segment smoothly transited to neck portion 200 and a tip for incising the trabecular meshwork of the eye.

In the present embodiment, the head portion 100 has a total length of 0.8 mm, and the cylindrical segment thereof has a diameter of 0.5 mm. However, in the context of the present invention, the total length of the head portion 100 is 0.6-1.0 mm, and the diameter of cylindrical segment of the head portion 100 is 0.4-0.6 mm.

The neck portion 200 is preferably in form of an elongated shape with a length L1 of 26 mm. However, in the context of the present invention, the length of the neck portion 200 is 22-30 mm. The front end of the neck portion 200 is connected to the head portion 100, the rear end of the neck portion 200 is connected to the handle portion 300, and the diameter of the neck portion 200 is gradually increased from the front end to the rear end thereof. In this easy way, the operator can conveniently exert force to incise the trabecular meshwork of the eye.

The handle portion 300 preferably has a length L2 of 80 mm. However, in the context of the present invention, the length of the handle portion is 70-130 mm. According to the present invention, the handle portion 300 has an operation part 320 in the middle thereof and a joining part 310 at least one end of the operation part 320. The joining part 310 is connected to the rear end of the neck portion 200. The operation part 320 is preferably in form of cylinder and the joining part 310 is preferably in form of circular truncated cone. The end of the joining part 310 connected to the neck portion 200 is smaller than the other end thereof.

In the present embodiment, for increasing the availability of the GT knife, both ends of the handle portion 300 respectfully have a neck portion 200 and a head portion 100. And each neck portion 200 is in form of an elongated shape.

The operation part 320 may be provided with a plurality of projections 321, and a plurality of threaded structures 322 are provided between the adjacent projections 321. In the present embodiment, there are projections 321 in the middle and both ends of the operation part 320, and the length of the projection 321 in the middle portion is longer than that of the projections 321 at the ends. To increase the friction in relation to the hands of the operator, threaded structures 322 are arranged between the projection 321 in the middle portion and the projections 321 at the ends. The projections 321 and the threaded structures 322 may be formed by rubber sleeves or rubber rings sleeved on the operation part 320, or formed by stainless-steel material integrally formed with the operation part 320. Preferably, the projections 321 and the threaded structures 322 are integrally formed with the operation part 320.

It should be noted that, for that the neck portion can be displaced for incising the trabecular meshwork of the eye at different situation, the joining part 310 may be detachably connected to the operation part 320, namely, the neck portion 200 is detachably connected to the operation part 320. Particularly, the joining part 310 is connected to the operation part 320 by screws. In this case, the joining part 310, the neck portion 200 and the head portion 100 may be integrally formed.

Referring to FIG. 2, the neck portion 200 of the GT knife in this embodiment is designed in a bending shape, including a first transition part 210 connected to the head portion 100 and a second transition part 220 connected to the handle portion 300. The first transition part 210 and the second transition part 220 forms a bending angle, especially 135 degrees. However, in the context of the present invention, the bending angle is 125-145 degrees.

The second transition part 220 is preferably in a line with the handle portion 300. The first transition part 210 may be upwardly or downwardly bended relative to the second transition part 220, and the head portion 100 is inclined relative to the first transition part 210 at an angle of 85-95 degrees. In this embodiment, there are neck portion 200 and head portion 100 at both ends of the handle, and the first transition part 210 of the neck portion 200 at one end of the handle is upwardly bended relative to the second transition part 220 thereof, while the first transition part 210 of the neck portion 200 at the other end of the handle is downwardly bended relative to the second transition part 220 thereof.

In this embodiment the first transition part 210 of the neck portion 200 has a length L11 of 11 mm, the second transition part 220 of the neck portion 200 has a length L12 of 15 mm, and the neck portion 200 has diameter gradually increased from the first transition part 210 to the second transition part 220. However, in the context of the present invention, the length of the first transition part 210 is 9-13 mm, the length of the second transition part 220 is 13-17 mm.

The handle portion 300 has a length L2 of 80 mm in the present embodiment. However, in the context of the present invention, the length of the handle portion is 70-130 mm.

Obviously, the above-mentioned embodiments of the present invention are only examples for clearly illustrating the technical solution of the present invention, rather than limiting the embodiments of the present invention. Any modifications, equivalent replacements and improvements made within the spirit and principles of claims of the present invention shall be included within the protection scope of the claims of the present invention.

What's claimed is:

1. A knife for GT, comprising a handle portion, at least one end of the handle portion being sequentially connected to a neck portion and a head portion, wherein the neck portion is smoothly transited to the head portion and formed an angle of 85-95 degrees therebetween, the head portion has a blunt tip end, and a diameter of the head portion is decreased towards the blunt tip end.

2. The knife for GT according to claim 1, wherein the head portion is designed into a cylindrical shape with the blunt tip end, including a cylindrical segment smoothly transited to the neck portion.

3. The knife for GT according to claim 2, wherein a total length of the head portion ranges from 0.6 mm to 1.0 mm.

4. The knife for GT according to claim 2, wherein a diameter of the cylindrical segment of the head portion ranges from 0.4 mm to 0.6 mm.

5. The knife for GT according to claim 1, wherein the neck portion is preferably in form of an elongated shape with a length of 22-30 mm.

7. The knife for GT according to claim 5, wherein both ends of the handle portion are respectively sequentially connected to the neck portion and the head portion.

8. The knife for GT according to claim 1, wherein a front end of the neck portion is connected to the head portion, a rear end of the neck portion is connected to the handle portion, and a diameter of the neck portion is gradually increased from the front end to the rear end thereof.

9. The knife for GT according to claim 1, wherein the neck portion is designed in a bending shape, including a first transition part connected to the head portion and a second transition part connected to the handle portion, with the first transition part and the second transition part forming a bending angle of 125-145 degrees.

10. The knife for GT according to claim 9, wherein a length of the first transition part is 9-13 mm.

11. The knife for GT according to claim 9, wherein a length of the second transition part is 13-17 mm.

12. The knife for GT according to claim 9, wherein a diameter of the neck portion is gradually increased from the first transition part to the second transition part.

13. The knife for GT according to claim 9, wherein both ends of the handle portion are respectively sequentially connected to the neck portion and the head portion.

14. The knife for GT according to claim 1, wherein a length of the handle portion is 70-130 mm.

15. The knife for GT according to claim 1, wherein the handle portion includes an operation part for grabbing and a joining part connected to the neck portion, the operation part is in form of cylinder and the joining part is in form of circular truncated cone with an end of the joining part connected to the neck portion is smaller than the other end thereof.

16. The knife for GT according to claim 15, wherein the operation part is provided with a plurality of projections.

17. The knife for GT according to claim 16, wherein a plurality of threaded structures are provided between the adjacent projections.

18. The knife for GT according to claim 16, wherein the joining part is detachably connected to the operation part.

19. The knife for GT according to claim 18, wherein the joining part, the neck portion and the head portion are integrally formed.

20. A use of the knife for GT according to claim 1 in a surgery treating glaucoma.
